(19) United States
(12) Patent Application Publication
(10) Pub. No.: US 2011/0065962 A1
(43) Pub. Date: Mar. 17, 2011

(54) PROCESS FOR THE PREPARATION OF AROMATIC ALPHA-HYDROXY KETONES

(76) Inventors: Enzo Meneguzzo, Sesto Calende (IT); Gabriele Norcini, Comabbio (IT); Giuseppe Li Bassi, Gavirate (IT)

(21) Appl. No.: 12/993,241

(22) PCT Filed: May 7, 2009
(86) PCT No.: PCT/EP2009/055518

§ 371 Date: Nov. 17, 2010

(30) Foreign Application Priority Data
May 9, 2008 (IT) VA2008A000027

(51) Int. Cl. C07C 45/00

(52) U.S. Cl. 568/323

(57) ABSTRACT

Process for the preparation of aromatic alpha-hydroxyketones (aromatic α-hydroxyketones) that does not require the use of chlorine, sulfuryl chloride or bromine and comprises the halogenation of an intermediate aromatic ketone with a hydrogen halide in the presence of an oxidising compound.

### FIELD OF THE INVENTION

The present invention relates to a process for the preparation of aromatic alpha-hydroxyketones (aromatic α-hydroxyketones) that does not require the use of chlorine, sulfuryl chloride or bromine.

### STATE OF THE ART

In the present text with the expression aromatic α-hydroxyketones we mean ketones wherein one of the susbstituent of the carbon atom of the carbonyl group is an aryl group and the other is an alkyl group bearing a hydroxyl (—OH) on the carbon atom which is adjacent to the carbonyl group.

Aromatic α-hydroxyketones are widely used as photoinitiators. The more common synthetic pathway leading to aromatic α-hydroxyketones comprises as a key intermediate the α-haloketone.

As it is reported in EP 3002 and in WO 2004099111, the α-haloketone is obtained from the reactions of alkyl aryl ketones with chlorine, bromine or sulfuryl chloride that proceed to the corresponding α-haloalkyl aryl ketones: the reported methods also require the use of halogenated organic solvents.

The utilization of bromine, sulfuryl chloride and chlorine involves disadvantages. If bromine is used, the cost is high; sulfuryl chloride implies specific plant facilities that can treat the reaction by-products, such as sulfurous anhydride.

As far as chlorine is concerned, which is classified among toxic gases, particular precautions are needed to ensure the safety of the process.

In Can. J. Chem. 68, 1990, a synthesis of α-hydroxy-isobutyrophenone from isobutyrophenone that does not require the use of chlorine or bromine is reported.

The reaction is however carried out with large excess of the reagents; for about 1 mmol of isobutyrophenone 100 mmol of sodium hydroxide and 900 mmol of potassium chloride in the presence of 3 mmol of sodium hypochlorite are used. The reaction takes 20 hours and has a 70% yield in α-hydroxy-isobutyrophenone. As it can be easily understood, the method is not applicable on industrial scale because of the huge volumes and the high excess of reagents that are involved. In the literature, examples of α-halogenation of aryl ketones that do not comprise the use of chlorinated organic solvents, but use instead ionic liquids, such as (BF₄)⁻ salts, are reported; on this issue, reference can be found, by way of example, in Synthetic Communications (2006), 36(6), 777-780.

Ionic liquids are however generally expensive and sensitive to humidity and their industrial use is rather troublesome.

Examples of halogenations by means of redox systems based on ipochlorite/chloride at acid pH of compounds bearing relatively reactive hydrogen atoms, such as those in benzyl position or in alpha to two keto groups are also known (by way of example from JP 10175891 and Tetrahedron Letters (2005), 46(28), 4749-4751).

References to redox systems based on hydrogen peroxide/HBr for several bromaination reactions are more numerous (by way of example, Synthetic Communications (2003), 33(8), 1399-1403).

A process for the preparation of aromatic α-hydroxyketones that does not require the use of chlorinated solvents, or of any other solvent, or the use of chlorine, sulfuryl chloride and bromine has now been found.

The present invention involves the in situ formation of halogenating compounds and provide the obtainment of intermediate and final product, even in the absence of solvent, without the above mentioned disadvantages.

As far as the Applicant knows, a process useful for the preparation of aromatic α-hydroxyketones based on the halogenating (and particularly chlorinating) redox systems here below detailed is not described in the literature, as well as a procedure for the production of aromatic α-hydroxyketones that does not require the use of any organic solvent for the preparation of the halogenated intermediate (aromatic α-halo ketone).

The procedure according to the invention is particularly suitable for the preparation of aromatic α-hydroxyketones bearing two alkyl groups (or a cycloalkyl group) in the α-position of the carbonyl group.

### DETAILED DESCRIPTION

It is therefore a fundamental object of the present invention a procedure for the preparation of aromatic α-hydroxy ketones and of bis aromatic α-hydroxy ketones comprising the following steps:
a) acylation of an aromatic compound of the formula

ArH
or of formula

HAr—Y—ArH,
wherein Y is simple bond, CH₂, O, S, CH═CH or NR⁰ with R⁰ C₁-C₁₂ linear or branched alkyl and Ar is an aryl group
with an acyl halide of the formula

XCOC(H)R¹R²
wherein X is Br or Cl and R¹ and R² are, independently, a C₁-C₁₂ linear or branched alkyl group which is unsubstituted or substituted with —OH, alkoxyl, aryl or —NR³R⁴, R³ and R⁴ being C₁-C₁₂ linear or branched alkyl groups or forming together a C₅-C₈cycloalkyl group; or R¹ e R² form together a C₅-C₈ cycloalkyl that may be substituted with —OH, alkoxyl, aryl, —NR³R⁴, R³and R⁴ being C₁-C₁₂ linear or branched alkyl groups or forming together a C₅-C₈ cycloalkyl group,
to obtain an aromatic ketone of the formula

ArCOC(H)R¹R²
or of the formula

R¹R²(H)CCOAr—Y—ArCOC(H)R¹R²,
wherein Ar, Y, R¹ e R² have the above detailed meaning;
b) halogenation of the aromatic ketone by means of reaction with a hydrogen halide HX in the presence of an oxidising compound, to obtain an aromatic α-halo ketone of the formula

ArCOC(X)R¹R²
or of the formula

R¹R²(X)CCOAr—Y—ArCOC(X)R¹R²,
wherein Ar, Y, X, R¹ e R² have the above detailed meaning;
c) hydroxylation of the α-halo ketone with an aqueous base to obtain the aromatic α-hydroxy ketone of the formula

ArCOC(OH)R¹R²
or of the formula

R¹R²(OH)CCOAr—Y—ArCOC(OH)R¹R²,
wherein Ar, Y, X, R¹ e R² have the above detailed meaning.

The procedure of the invention is of general applicability and provided several α-hydroxy ketones that are already known and used as photoinitiators; among those, the most interesting are reported here below:

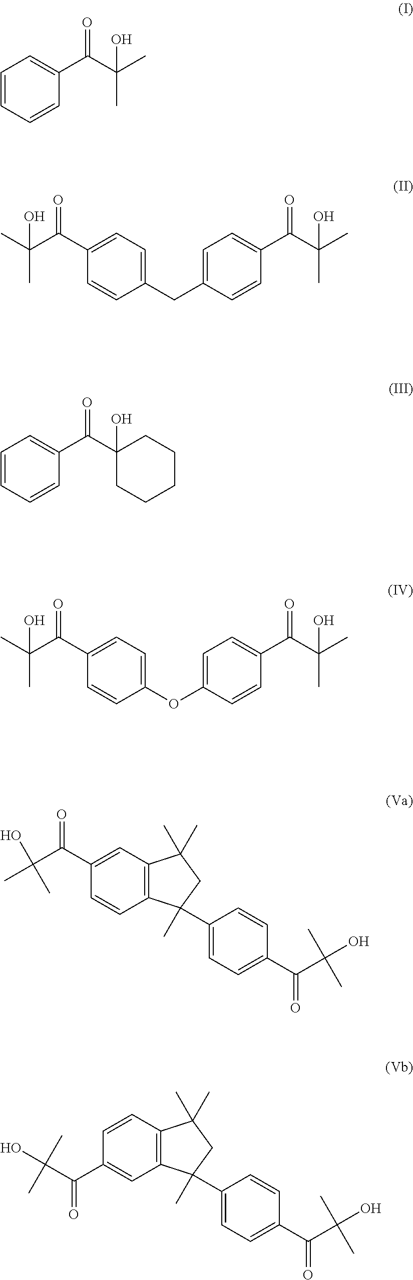

More in general, the procedure according to the invention is applicable to aromatic compounds of the formula ArH and HAr—Y—ArH, wherein Ar is phenyl, which may be unsubstituted or substituted with one or more C₁-C₁₂ alkyl groups, C₅-C₈cycloalkyl, C₁-C₄-haloalkyl, halogen; or Ar is substituted with a 1,1,3-trimethylindane group through a simple bond with the carbon atom 3 of the indane ring.

According to a particularly advantageous aspect of the invention, the procedure provides compounds containing two or more aromatic α-hydroxy-keto groups and specifically symmetric aromatic bis α-hydroxy ketones, by way of example, if the acylation reaction is carried out on an aromatic compound of the formula HAr—Y—ArH where Ar is unsubstituted phenyl and Y is O, S or CH₂ and R¹ and R² in the acyl halide are methyl.

According to another form of realization of the present invention, the aromatic compound has the formula ArH, wherein Ar is unsubstituted phenyl and R¹ and R² in the acyl halide are methyl, or together form a cyclohexyl group; or Ar is phenyl substituted with a 1,1,3-trimethylindane group and R¹ and R² in the acyl halide are methyl.

The acylation reaction of step a) is a Friedel Crafts acylation between the aromatic ArH or HAr—Y—ArH compound and an acyl halide of the formula XCOC(H)R¹R², wherein X is Cl or Br and R¹ and R² have the above detailed meaning; preferably, the acylation is catalyzed by aluminum trochloride and is carried out by reacting aluminum trichloride on the aromatic compound which is dissolved in an acyl chloride, without the use of any solvent.

The temperature during this step is usually kept between 0° and 60° C.

Step a) comprises, after the acylation, a final stage which is referred to as quenching or hydrolysis and is generally performed by trating the reaction mixture with a 4-10% wt HCl aqueous solution at temperature between 50 and 60° C.

At the end of the quenching, the catalyst is dissolved in the aqueous phase (quenching water) and the reaction product, the aromatic ketone, is separated from the aqueous phase and can be recovered and directed to the following step (step b)).

Alternatively, and according to a further advantageous embodiment of the invention, the quenching water that contains the catalyst and HCl, may be used as the aqueous medium where the following halogenations of step b) is performed. In this case, it may be necessary to regulate their content of hydrogen halide, in order to be in the right condition to directly continue with the halogenation, without separating the phases.

During step b) the hydrogen halide is preferably hydrogen chloride hydrogen bromide or Is prepared in situ by mixing sulfuric acid and a bromide or chloride of a alkaline metal salt.

When the hydrogen halide is hydrogen chloride or is prepared in situ by mixing sulfuric acid and an alkaline metal salt chloride, the reaction shall be carried out in a closed vessel at pressure between 0.5 and 3 bar.

Surprisingly, the best results are obtained by carrying out the halogenation of step b) without any organic solvent, on the aromatic ketone in liquid form and dispersed in an aqueous medium; in this way, it is possible to remarkably reduce the amount of reactants and, at the same time, to avoid the use of organic solvents, particularly of halogenated solvents, such as methylene chloride and dichlorobenzene.

The liquid form of the aromatic ketone may advantageously be obtained by operating at a temperature above its melting point.

Preferably, according to the procedure of the invention, an excess of hydrogen halide and oxidizing compound is used, the molar ratio between oxidizing compound and aromatic ketone ranging between 1.1:1 to 10:1 and the molar ratio between hydrogen halide and aromatic ketone ranging from 1.1:1 and 20:1.

The inexpensiveness of the reactants, the possibility of operating without any organic solvent and the simplicity of the procedure, that avoids the use of chlorine, bromine or sulfuryl chloride, largely compensate a possible limited excess of hydrogen halide and oxidising compound.

According to a particularly advantageous embodiment, step a), step b) and step c) are carried out in the absence of organic solvent, the aromatic compound and the aromatic ketone being in liquid form, dispersed in an aqueous medium.

The temperature of the halogenation reaction is preferably comprised between 40° and 120° C.

Alkaline and alkaline-earth metal salts of hypochlorite and hydrogen peroxide may be used as the oxidizing compounds.

Hydrogen peroxide is preferably used as a 33% aqueous solution.

The preferred oxidising compounds are sodium ipochlorite and calcium ipochlorite. Sodium ipochlorite can be directly used in step b) in its most common commercial form , that is as a 10-13% wt aqueous solution.

Calcium ipochlorite is commercially available as a solid with about 65% chlorine active matter; for use in step b), it can be diluted in water in advance or it can be directly added to the aqueous medium in which step b) is performed.

Chloride of lime can also be used as the calcium ipochlorite source in the procedure of the invention.

The oxidizing compounds of step b), are used in the form of aqueous solution with concentrations ranging from 0.5 and 4 mol/l.

The hydrogen halide is normally used in step b) in aqueous solution, preferably with concentrations ranging from 3 to 14 mol/l.

When the halogenations is carried out with alkaline metal halides, it preferable to add in the aqueous medium for 4 to 6 moles of sulfuric acid per mole of aromatic ketone.

Advantageously, the procedure of the present invention can be used for the preparation of α-hydroxy ketones via α-chloro ketones; the latter intermediates are preferred in the synthesis of aromatic α-hydroxy ketones, because they allow to completely avoid the use of bromine derivatives.

For this reason, the hydrogen halide is more preferably hydrogen chloride, or it is prepared in situ by mixing sulfuric acid with an alkaline metal salt chloride, such as sodium chloride.

In Table 1, some useful conditions that can be used to successfully conduct the reaction of step b) are reported.

TABLE 1

| | Hydrogen halide | Oxidising compound | |
|---|---|---|---|
| Method | (mol/mol of ketone) | (mol/mol ketone) | Temp. ° C. |
| A | HCl 37% | NaClO 12.5% | 40°-100° C. |
| | (1-10) | (1-2) | |
| B | HCl 37% | Ca(ClO)₂ 65% | 40°-100° C. |
| | (1-6) | (1-2) | |
| C | HCl 37% + H₂SO₄ 64% | H₂O₂ 33% | 40°-100° C. |
| | (6-12 + 4-8) | (5-10) | |
| D | NaCl + H₂SO₄ 64% | H₂O₂ 33% | 60°-120° C. |
| | (4-16 + 4-16) | (2-6) | |
| E | NaCl + H₂SO₄ 64% | NaClO 12.5% | 60°-120° C. |
| | (4-16 + 4-16) | (2-6) | |
| F | HBr 48% | H₂O₂ 33% | 50°-80° C. |
| | (2-4) | (1-4) | |
| G | HBr 48% | NaClO 12.5% | 50°-80° C. |
| | (2-4) | (1-4) | |

The final step of the procedure of the invention is the reaction of the α-halo ketone that is obtained at the end of step b) with an aqueous alkali, preferably with sodium, barium or potassium hydroxide at concentration from 5 to 50% wt in water, preferably without any organic solvent and in the presence of a phase transfer catalyst, such as benzyl trimethylammonium chloride.

The reaction of step c) is a substitution reaction, the α-halogen atom being replaced by an —OH group; the reaction can be performed on the crude α-halo ketone which is obtained from step b).

At the end of the reaction, the α-hydoxy ketone can be recovered by separating the phases, washing it with water and possibly purificating it by means of the usual industrial methods, such as by distillation or crystallization.

The procedure according to the invention provides the α-hydoxy ketone with high yield form the corresponding aromatic compound, as it is apparent from the following examples.

### EXAMPLES

### Example 1

Preparation of 2-hydroxy-2-methyl-propiophenone

a) Acylation
Synthesis of 2-methyl-propiophenone (isobutyrophenone)

123 g of aluminum chloride (1.02 moles) were added in portions in two hours to a solution of 120 g of benzene (1.53 moles) and 108.2 g of isobutyryl chloride (1.02 moles) under stirring keeping the temperature at 5° C. The mixture was maintained under stirring for an additional hour without cooling. The reaction was checked by TLC (SiO₂, toluene). The mixture was poured in ice under stirring. The organic layer was separated and the solvent evaporated under vacuum and the product was distilled at 163° C., 160 mmHg obtaining 140 g of colorless oil (95% yield) that was used for the next steps.

b) Halogenation
Synthesis of 2-chloro-2-methyl-propiophenone (Method A).

31 g of NaClO 12% water solution (0.05 moles) were dropped in 90′ to a stirred suspension of 7.4 g of the 2-methyl-propiophenone obtained in step a) (0.05 moles) in 11.84 g of hydrochloric acid 37% (0.12 moles) at 40° C. The temperature rose to 57° C. The suspension was stirred for another hour. After cooling, the organic phase was separated and checked by TLC (SiO₂, toluene) observing a conversion greater than 85%. The organic phase (oil) was used for the next steps without further purification.
Synthesis of 2-bromo-2-methyl-propiophenone (Method G).

31 g of NaClO 12% water solution (0.05 moles) were dropped in 90′ to a stirred suspension of 7.4 g of the 2-methyl-propiophenone obtained in step a) (0.05 moles) in 20.23 g of hydrobromic acid 48% (0.12 moles) at 20° C. The temperature rose to 50° C. The suspension was stirred for 12 hours. After cooling, the organic phase was separated and checked by TLC (SiO₂, toluene) observing a conversion greater than 95%. The organic phase (oil) was used for the next steps without further purification.

c) Hydroxylation
Synthesis of 2-hydroxy-2-methyl-propiophenone.

Aliquots of the oil obtained in step b) according to Method A or Method G corresponding to 20 mmoles were heated under stirring from 40° to 80° C. in the presence of NaOH 50% (25 mmoles) and benzyltriethylammonium chloride (0.025 mmoles). After 60′, TLC (SiO₂, tolene/methanol 85/15) indicated that the reaction was complete. The organic phase was separated and distilled under vacuum (182° C., 160 mmHg) obtaining 2.95 g of product (90% yield).

H¹NMR (300 MHz, CDCl₃): δ: 7.96-8.04 (m,2H); 7.53-7.60 (m,1H); 7.42-7.50 (m,2H); 1.65 (s,6H).

### Example 2

Preparation of a mixture of 2-hydroxy-1-{3-[4-(2-hydroxy-2-methyl-propionyl)-phenyl]-1,1,3-trimethyl-indan-5-yl}-2-methyl-propan-1-one and 2-hydroxy-1-{1-[4-(2-hydroxy-2-methyl-propionyl)-phenyl]-1,3,3-trimethyl-indan-5-yl}-2-methyl-propan-1-one.
a) Acylation
   Synthesis of a mixture of 1-[4-(5-isobutiryl-1,3,3-trimethyl-indan-1-yl)-phenyl]-2-methyl-propan-1-one and 1-[4-(6-isobutiryl-1,3,3-trimethyl-indan-1-yl)-phenyl]-2-methyl-propan-1-one

14.66 g of aluminum chloride (110 moles) were added in portions in one hour to a solution of 11.82 g of 1,3,3-trimethyl-1-phenyl-indane (50 mmoles) and 13.38 g of isobutyryl chloride (123 mmoles) under stirring keeping the temperature at 25° C. The mixture was heated and maintained at 60° C. under stirring for an additional hour; the viscosity of the mixture increased. The reaction was checked by TLC (SiO₂, toluene). The mixture was treated with 112 g of hydrochloric acid 4%, not exceeding 80° C. The organic layer separated at 60° C. as a light oil that was used without purification for the next step.
b) Halogenation.
   Synthesis of a mixture of 2-chloro-1-{3-[4-(2-chloro-2-methyl-propionyl)-phenyl]-1,1,3-trimethyl-indan-5-yl}-2-methyl-propan-1-one and 2-chloro-1-{1-[4-(2-chloro-2-methyl-propionyl)-phenyl]-1,3,3-trimethyl-indan-5-yl}-2-methyl-propan-1-one. (Method A)

1 g of the oil obtained in the step a) (2.66 mmoles) was suspended under stirring in 5.2 g of hydrochloric acid 37% (52.7 mmoles) at 100° C. in a pressure reactor. 3.8 g of NaClO 12.5% (6.4 mmoli) were added in 1 hour. The mixture is stirred at 100° C. for an additional hour. After TLC (Si₂, toluene) control, the mixture was cooled and the organic phase was collected after separation from the water phase. The oil so obtained (1 g) was used for the next step.
Synthesis of a mixture of 2-chloro-1-{3-[4-(2-chloro-2-methyl-propionyl)-phenyl]-1,1,3-trimethyl-indan-5-yl}-2-methyl-propan-1-one and 2-chloro-1-{1-[4-(2-chloro-2-methyl-propionyl)-phenyl]-1,3,3-trimethyl-indan-5-yl}-2-methyl-propan-1-one. (Method B)

0.28 g of the oil obtained in the step a) (0.74 mmoles) were suspended under stirring in 0.88 g of hydrochloric acid 37% (8.9 mmoles) at 50° C. in a pressure reactor. Then 0.36 g of Ca (ClO)₂ 65% (1.64 mmoli) were added. The mixture is stirred at 60° C. for one hour. After TLC (SiO₂, toluene) control, the mixture was cooled and the organic phase was collected after separation of the water phase. The oil so obtained (0.3 g) was used for the next step.
c) Hydroxylation.
   Synthesis of a mixture of 2-hydroxy-1-{3-[4-(2-hydroxy-2-methyl-propionyl)-phenyl]-1,1,3-trimethyl-indan-5-yl}-2-methyl-propan-1-one and 2-hydroxy-1-{1-[4-(2-hydroxy-2-methyl-propionyl)-phenyl]-1,3,3-trimethyl-indan-5-yl}-2-methyl-propan-1-one.

0.3 g of the oil obtained according method A or B (0.67 mmoles) were stirred at reflux with 0.32 g of NaOH 30% (2.42 mmoles) in the presence of 0.04 g of benzyl-triethylamonium chloride. After two hours the reaction was complete (TLC SiO₂, toluene/methanol 85/15). After standing at 60° C. the light organic phase was collected and washed two times with 5 ml of water. The product was obtained as an oil (0.24 g, 87%).

H1NMR(300 MHz, CDCl₃):δ: 7.9-8.1 (m, 3H); 7.8 (s, 1 H); 7.2-7.4 (m, 3H); 4.1-4.2 (m, 2H); 2.4-2.5 (d, 1H); 2.2-2.3 (d,1H); 1.6-1.8 (m,15H); 1.4 (m,3H); 1.1 (m,3H).

### Example 3

Preparation of 2-hydroxy-1-{4-[4-(2-hydroxy-2-methyl-propionyl)-phenoxy}-2-methyl-1-propane-1-one.
a) Acylation
   Synthesis of 1-[4-(4-isobutyryl-phenoxy)-phenyl]-2-methyl-propane-1-one.

15.33 g of aluminum chloride 115 mmoles) were added in portions in one hour to a solution of 8.6 g of diphenylether (50 mmoles) and 11.97 g of isobutyryl chloride (110 mmoles) under stirring keeping the temperature between 5° and 15° C. The mixture was maintained under stirring for an additional hour at 15° C. then heated at 50° C. for one hour. The mixture was treated with 100 ml of water under stirring. The organic layer was separated obtaining 11 g of yellow oil that was used for the next step without purification. A sample was crystallized from petroleum ether 40°-65° C. affording a whitish solid with mp 54° C.

H1NMR(300 MHz, CDCl₃):δ: 7.98 (d, 4H); 7.04 (d, 4H); 3.45-3.55(m, 2H); 1.21 (d, 12H).
b) Halogenation
   Synthesis of 2-chloro-1-{4-[4-(2-chloro-2-methyl-propionyl)-phenoxy]-phenyl}-2-methyl-propan-1-one. (Method C)

15.2 g of hydrogen peroxide 33% (148 mmoles) were added In a pressure vessel to a suspension of 3.03 g of 1-[4-(4-isobutyryl-phenoxy)-phenyl]-2-methyl-propane-1-one (9.8 mmoles) in 21.67 g of HCl 37% (220 mmoles) and 18 g of sulfuric acid 64% (118 mmoles). Then the mixture was heated at 120° C. under stirring in pressure. After 40′ the reaction was cooled and checked by TLC (SiO2, toluene) observing the almost complete transformation of the starting material. The organic phase was used in the next step without further purification.
Synthesis of 2-chloro-1-{4-[4-(2-chloro-2-methyl-propionyl)-phenoxy]-phenyl}-2-methyl-propan-1-one. (Method D)

5.06 g of hydrogen peroxide 33% (49 mmoles) were added in a pressure vessel to a suspension of 3.03 g of 1-[4-(4-isobutyryl-phenoxy)-phenyl]-2-methyl-propane-1-one (9.8 mmoles) in 17.4 g of sulfuric acid 64% (114 mmoles)and 6.84 g of NaCl (117 mmoles). Then the mixture was heated at 120° C. under stirring and pressure. After 40′ the reaction was cooled and checked by TLC (SiO2, toluene) observing the almost complete transformation of the starting material. The organic phase was collected by filtration and used in the next step without further purification.
Synthesis of 2-chloro-1-{4-[4-(2-chloro-2-methyl-propionyl)-phenoxy]-phenyl}-2-methyl-propan-1-one. (Method E)

12 g of NaClO 12% (20.1 mmoles) were added in 15′ In a pressure vessel to a stirred suspension of 1.5 g of 1-[4-(4-isobutyryl-phenoxy)-phenyl]-2-methyl-propane-1-one (9.8 mmoles) and 6.84 g of NaCl (117 mmoles) in 15 g of sulfuric acid 64% (98 mmoles) at 70° C. After one hour in the same conditions the reaction was complete(TLC SiO₂, toluene). The separated organic phase was used in the next step without further purification.
Synthesis of 2-bromo-1-{4-[4-(2-bromo-2-methyl-propionyl)-phenoxy]-phenyl}-2-methyl-propan-1-one. (Method F)

2.21 g of hydrogen peroxide 33% (21.5 mmoles) were added in 20′ to a stirred suspension of 3.03 g of 1-[4-(4-isobutyryl-phenoxy)-phenyl]-2-methyl-propane-1-one (9.8 mmoles) in 7.04 g of hydrobromic acid 48% (41.8 mmoles) at 20° C. Then the mixture was heated at 70° C. for one hour. The reaction was checked by TLC (SiO₂, toluene). The whole mixture was used for the next step without purification.
Synthesis of 2-bromo-1-{4-[4-(2-bromo-2-methyl-propionyl)-phenoxy]-phenyl}-2-methyl-propan-1-one. (Method G)

13.73 g of NaClO 12% (23 mmoles) were slowly added in 20′ to a stirred suspension of 3.03 g of of 1-[4-(4-isobutyryl-phenoxy)-phenyl]-2-methyl-propane-1-one (9.8 mmoles) in 7.04 g of hydrobromic acid 48% (41.2 mmoles) at 45° C. Then the mixture was heated at 60° C. for 20′. The reaction was checked by TLC (SiO₂, toluene). The whole mixture was used for the next step without purification.
c) Hydroxylation
   Synthesis of 2-hydroxy-1-{4-[4-(2-hydroxy-2-methyl-propionyl)-phenoxy]-phenyl}-2-methyl-propan-1-one (from the di-chloro intermediate)

The di-chloro intermediate prepared according to Method D was dissolved in 10.61 g of i-propyl alcohol and 2.6 g of water. 2.3 g of NaOH 50% were added to the so obtained solution and after 15′ at 80° C. the reaction was complete (TLC SiO₂, toluene/methanol 85/15). After cooling and dilution with 16.65 g of water the pH was adjusted at 3 with conc. HCl. The reaction product separates as a white solid, 2.3 g were collected by filtration (68%), mp 97°-99° C. H1NMR(300 MHz, CDCl₃): δ: 8.10 (d,4H); 7.07 (d,4H); 3.9 (s,2H);1.63 (s,12H).
Synthesis of 2-hydroxy-1-{4-[4-(2-hydroxy-2-methyl-propionyl)-phenoxy]-phenyl}-2-methyl-propan-1-one (from the di-bromo intermediate)

The suspension of the di-bromo intermediate obtained according to Method F or G was stirred with 3 g of a Na₂S₂O₅ 10% water solution at 85° C. for 10′, then diluted with 10.6 g of i-propyl alcohol and 2.6 g of water. To the so obtained solution 2.3 g of NaOH 50% were added and after 15′ at reflux the reaction was complete (TLC SiO₂, toluene/methanol 85/15). After cooling and dilution with 13.3 g of water the pH was adjusted at 3 with conc. HCl. The reaction product separate as a white solid, 3 g were collected by filtration (90%), mp 97°-99° C. H1NMR(300 MHz, CDCl₃): δ: 8.10 (d,4H); 7.07 (d,4H); 3.9 (s,2H);1.63 (s,12H).

### Example 4

Preparation of 1-hydroxy-cyclohexyl-phenylketone.

a) Acylation
Synthesis of cyclohexyl-phenylketone.

13.7 g of aluminum chloride (103 mmoles) were added in portions in 1 hour to a stirred solution of 15 g of the cyclohexanecarboxylic acid chloride (100 mmoles) in 24 g of benzene at 10-15° C. The mixture was then heated at 60° C. for 20′ to complete the reaction. The mixture was cooled to room temperature and poured in 100 ml of water. The organic phase was separated and the solved distilled off under vacuum affording 18.6 g of an oil.

H1 NMR(300 MHz, CDCl₃): δ: 7.90-8.00 (d,2H); 7.40-7.60 (m,3H); 3.20-3.35 (m,1H); 1.70-2.00 (m,5H); 1.20-1.60 (m,5H).
b) Halogenation
   Synthesis of 1-bromo-cyclohexyl-phenylketone (Method G).

18.7 g of NaClO 12% (34.8 mmoles) were added at 60° C. in 30′ to a stirred dispersion of 4.71 g of cyclohexyl-phenylketone (25 mmoles) in 11.52 g of hydrobromic acid 48% (68.3 mmoles). The mixture was then heated from 60° to 100° C. in two hours. After cooling at 70° C. the organic phase was separated and washed with 50 g of a 10% water solution of sodium sulfite, then with 50 g of water. The organic phase (6.6 g) was used without purification for the next step.

H1NMR(300 MHz, CDCl₃): δ: 8.02-8.12 (d,2H); 7.38-7.60 (m,3H); 2.27-2.42 (m,2H); 2.10-2.25 (m,2H); 1.75-1.90 (m,2H); 1.47-1.65 (m,3H); 1.35-1.46 (m,1H).
Synthesis of 1-chloro-cyclohexyl-phenylketone (Method B).

4.01 g of Ca (ClO)₂ 65% (18.2 mmoles) were added in 60′ to a pressure vessel containing 1.88 g of cyclohexyl-phenylketone (10 mmoles) dispersed at 60° C. in 4.96 g of HCl 37% (50 mmoles). After 30′ under stirring at 60° C. the organic phase was separated and washed with 10 ml of water obtaining 10 g of an oil. The oil was used without purification for the next step (2.3 g).

H1NMR(300 MHz, CDCl₃): δ: 8.05-8.15 (d,2H); 7.38-7.60 (m,3H); 2.07-2.30 (m,4H); 1.75-1.90 (m,2H); 1.50-1.67 (m,3H); 1.25-1.4 (m,1H).
c) Hydroxylation
   Synthesis of 1-hydroxy-cyclohexyl-phenylketone.

6.6 g of 1-bromo-cyclohexyl-phenylketone obtained with Method G (24.7 mmoles), were dispersed in 6 g of NaOH 30% and heated to 80° C.; 100 mg of benzyl-triethylammonium chloride were added in two portions, and the mixture was stirred at 80° C. for one hour. The organic phase was separated and washed warm with 10 ml of water set a pH 3 with concentrated HCl. The organic phase crystallized from petroleum ether 40°-65° C. obtaining 3 g of a whitish solid (59%), mp 45°-46° C.

H1NMR(300 MHz, CDCl₃): 8: 7.97-8.07 (d,2H); 7.40-7.60 (m,3H); 3.45 (s, 1H); 1.97-2.12 (m,2H); 1.60-1.87 (m,7H); 1.25-1.45 (m,1H).

1. Procedure for the preparation of aromatic α-hydroxy ketones and of bis aromatic α-hydroxy ketones comprising the following steps:
a) acylation of an aromatic compound of the formula
ArH
or of formula
HAr—Y—ArH,
wherein Y is simple bond, CH₂, O, S, CH═CH or NR⁰ with R⁰ C₁-C₁₂ linear or branched alkyl and Ar is an aryl group with an acyl halide of the formula
XCOC(H)R¹R²
wherein X is Br or Cl and R¹ and R² are, independently, a C¹-C¹² linear or branched alkyl group which is unsubstituted or substituted with —OH, alkoxyl, aryl or —NR³R⁴, R³ and R⁴ being C₁-C₁₂ linear or branched alkyl groups or forming together a C₅-C₈ cycloalkyl group; or R¹ e R² form together a C₅-C₈ cycloalkyl that may be substituted with —OH, alkoxyl, aryl, —NR³R⁴, R³, and R⁴ being C₁-C₁₂ linear or branched alkyl groups or forming together a C₅-C₈ cycloalkyl group, to obtain an aromatic ketone of the formula
ArCOC(H)R¹R²
or of the formula
R¹R²(H)CCOAr—Y—ArCOC(H)R¹R²,
wherein Ar, Y, R¹ e R² have the above detailed meaning;
b) halogenation of the aromatic ketone by means of reaction with a hydrogen halide HX in the presence of an oxidising compound, to obtain an aromatic α-halo ketone of the formula
ArCOC(X)R¹R²
or of the formula
R¹R²(X)CCOAr—Y—ArCOC(X)R¹R²,
wherein Ar, y, X, R¹ e R² have the above detailed meaning;
c) hydroxylation of the α-halo ketone with an aqueous base to obtain the aromatic α-hydroxy ketone of the formula
ArCOC(OH)R¹R²
or of the formula
R¹R²(OH)CCOAr—Y—ArCOC(OH)R¹R²,
wherein Ar, Y, X, R¹ e R² have the above detailed meaning.

2. Procedure according to claim 1, wherein is Ar is phenyl, which may be unsubstituted or substituted with one or more C¹-C¹² alkyl groups, C₅-C₈ cycloalkyl, C₁-C₄-haloalkyl, halogen; or Ar is substituted with a 1,1,3-trimethylindane group through a simple bond with the carbon atom 3 of the indane ring.

3. Procedure according to claim 2 wherein the aromatic compound has the formula ArH and Ar is unsubstituted phenyl and R¹ and R² are methyl, or together form a cyclohexyl group; or Ar is phenyl substituted with a 1,1,3-trimethylindane group and R¹ and R² are methyl.

4. Procedure according to claim 2 wherein the aromatic compound has the formula HAr—Y—ArH where Ar is unsubstituted phenyl and Y is O, S or CH₂ and R¹ and R² are methyl.

5. Procedure according to claim 1, wherein the hydrogen halide is hydrogen chloride or hydrogen bromide, or is prepared in situ by mixing sulfuric acid with an alkaline metal salt of chlorine or bromine, and the oxidizing compound is an alkaline or alkaline-earth metal salts of hypochlorite or hydrogen peroxide.

6. Procedure according to claim 5, wherein the oxidizing compound is sodium hypochlorite or calcium hypochlorite.

7. Procedure according to claim 5, wherein the hydrogen halide is hydrogen chloride or is prepared in situ by mixing sulfuric acid with an alkaline metal salt of chlorine.

8. Procedure according to claim 1, wherein step b) is carried out in the absence of organic solvent on the aromatic ketone in liquid form, dispersed in an aqueous medium.

9. Procedure according to claim 8, wherein step a) and step c) are carried out in the absence of organic solvent, the aromatic compound and the aromatic ketone being in liquid form, dispersed in an aqueous medium.

10. Procedure according to claim 1 wherein the molar ratio between oxidizing compound and aromatic ketone ranging between 1.1:1 to 10:1 and the molar ratio between hydrogen halide and aromatic ketone ranging from 1.1:1 and 20:1.

11. Procedure according to claim 8, wherein the acylation reaction is catalyzed by aluminum trichloride and comprises a final hydrolysis stage which is performed by treating the reaction mixture with a 4-10% wt HCl aqueous solution at the end of which the aluminum trichloride is dissolved in the aqueous phase (quenchin water) and the acylated reaction product separates from the aqueous phase.

12. Procedure according to claim 11 wherein the quenching water is used as the aqueous medium of step b).
